(19) United States
(12) Patent Application Publication
(10) Pub. No.: US 2005/0202446 A1
(43) Pub. Date: Sep. 15, 2005

(54) Methods for biopolymer sequencing using metal inclusions

(76) Inventors: Dan-Hui Dorothy Yang, Sunnyvale, CA (US); Ganapati R. Mauze, Sunnyvale, CA (US)

Correspondence Address: AGILENT TECHNOLOGIES, INC.;Intellectual Property Administration, Legal Department, DL429, P.O. Box 7599, Loveland, CO, 80537-0599, US

(21) Appl. No.: 10/798,062

(22) Filed: Mar. 11, 2004

(51) Int. Cl. C12Q 1/68; C12M 1/34

(52) U.S. Cl. 435/6; 435/287.2

(57) ABSTRACT

The invention provides a method for detection of a biopolymer. The biopolymer may include nucleic acids such as DNA or RNA that hybridize upon association with other molecules. The method of the invention teaches the steps of adding a metal to a biopolymer to form an initial complex and ramping a voltage through the initial complex to produce a detectable signal.

### TECHNICAL FIELD OF THE INVENTION

The invention relates to the field of biopolymers and to the detection and/or sequencing of biopolymers. More particularly the invention relates to the use of nanopores to sequence M-DNA and determine single base mismatch in DNA complexes.

### BACKGROUND OF THE INVENTION

Recently, Palok Aich and Jeremy Lee, international patent application WO 99/31115 have described a technique for converting a hybridized double strand of DNA into an electrically conductive wire called M-DNA. This invention teaches methods and devices for using M-DNA for the purpose of hybridization detection and sequence determination based on fluorescence quenching and electrochemical detection. These techniques are important to biochemists who are interested in sequencing and identifying various types of biopolymers. The technology, however, lacks identification specificity and in certain instances will only provide nonspecific binding with limited chemical doping control. Also, this technology requires labeling probe polynucleotide and/or target polynucleotide. Thus this is prone to be time consuming and require higher cost.

Nanotechnology is a developing field of interest in the life sciences and semiconductor industry. A number of nanopore structures have been proposed. However, many of these inventions suffer from limitations of stability of the pore and reproducibility of sequencing. Therefore, research is ongoing to develop nanopore structures that are stable and produce reproducible results.

It has been demonstrated that a voltage gradient can drive single-stranded charged biopolymers through a transmembrane channel, or nanopore (See Kasianowicz et al., “Characterization of individual polynucleotide molecules using a membrane channel”, Proc. Natl. Acad. Sci. USA, 93: 13770-13773, 1996). During the translocation process, the extended biopolymer molecule will block a substantial portion of the otherwise open nanopore channel. This blockage leads to a decrease in the ionic current flow of the buffer solution through the nanopore during the biopolymer translocation. The passage of a single biopolymer can be monitored by recording the translocation duration and the blockage current, yielding plots with predictable stochastic sensing patterns. From the uniformly controlled translocation conditions, the lengths of the individual biopolymers can be determined from the translocation time. Furthermore, it is desirable that the differing physical and chemical properties of the individual monomers of the biopolymer strand may generate a measurable and reproducible modulation of the blockage current that allows an identification of the specific monomer sequence of the translocating biopolymer. These initially proposed systems suffer from a number of problems. For instance, some of the proposed systems require the self-assembly of pore forming proteins on membranes (i.e. α-hemolysin). Reproducibility stability of protein membrane assembly and systems have been quite problematic. Secondly, commercial products require robustness not present in sensitive systems that require fluctuations of ionic currents for measurements. For these reasons, recent research has focused more on solid-state pore techniques that have an ability for high reproducibility and ease of fabrication. Such techniques as “ion beam sculpting” have shown some promise in fabricating molecular scale holes and nanopores in thin insulating solid-state membranes. These pores have also been effective in localizing molecular-scale electrical junctions and switches (See Li et al., “Ion beam sculpting at nanometer length scales”, Nature, 412: 166-169, 2001).

These techniques have shown similar consistent results and current blockage with double stranded DNA reminiscent of ionic current blockages observed when single stranded DNA are translocated through the channel formed by α-hemolysin in a lipid bilayer. The duration of these blockages have been on the millisecond scale and current reductions have been to 88% of the open-pore value. This is commensurated with translocation of a rod-like molecule whose cross-sectional area is 3-4 nm² (See Li et al., “Ion beam sculpting at nanometer length scales”, Nature, 412: 166-169, 2001). This methodology, however, suffers from the limitation that only crude measurements of the presence or absence of the translocating polymer can be made. In addition, these systems are incapable of actually determining the primary sequence (order of monomeric units) of the translocating biopolymer.

A second approach has been suggested for detecting a biopolymer translocating a nanopore in a solid-state material such as Si₃N₄. It is well known that the tunneling current has an exponential dependence upon the height and width of the quantum mechanical potential barrier to the tunneling process. This dependence implies an extreme sensitivity to the precise location in the pore of the translocating molecule. Both steric attributes and physical proximity to the tunneling electrode could cause changes in the magnitude of the tunneling current which would be far in excess of the innate differences expected between different base-types under ideal conditions for nucleotide sequencing. For this reason, it is difficult to expect the simplest tunneling configurations to have the specificity required to perform sequencing.

Resonant tunneling effects have been employed in various semiconductor devices including diodes and transistors. For instance, U.S. Pat. No. 5,504,347, Javanovic, et al., discloses a lateral tunneling diode having gate electrodes aligned with a tunneling barrier. The band structures for a resonant tunneling diode are described with a quantum dot situated between two conductors, with symmetrical quantum barriers on either side of the quantum dot. The resonant tunneling diode may be biased so that the energy level in the quantum dot matches the conduction band energy in one of the conductors. In this situation current versus applied voltage is at a local maximum. In addition, the resonant tunneling diode may be biased so that no energy level in the quantum dot matches the conduction band energy in either of the conductors. Current versus applied voltage is at a local minimum. As discussed previously, resonant tunneling electrodes will replace the fixed quantum dot with a mobile molecule such as a biopolymer. The tunneling barrier will occur across vacuum or liquid between the electrodes. These and other disclosed devices suffer from a few limitations. For instance, they must be capable of biopolymer identification in vacuum or in solution. In addition, the resonant tunneling electrodes must be constructed in a defined manner such that only one monomeric unit of the bipolymer may be identified or in position for resonant tunneling. For instance, if the biopolymer is DNA there is no guarantee that the DNA either entering or leaving the nanopore in the electrodes will be unraveled into a linear chain so that only one base, or base pair is in position for resonant tunneling. Secondly, for each of the nanopore technologies described above, there is a problem in distinguishing A and G, C and T since these nucleotides are similar in size and structure. This problem exists for both the natural and synthetic nanopore technologies. For these reasons, there is a need for improved systems and methods for sequencing biopolymers with high reproducibility and predictability. These and other problems with the prior art processes and designs are obviated by the present invention. The references cited in this application infra and supra, are hereby incorporated in this application by reference. However, cited references or art are not admitted to be prior art to this application.

### SUMMARY OF THE INVENTION

The invention provides a method for detecting and sequencing biopolymers. The method comprises, adding a metal to a biopolymer to form an initial complex, and applying a ramped voltage across a nanopore to the initial complex to produce a measurable signal.

### BRIEF DESCRIPTION OF THE DRAWINGS

Embodiments of the invention will now be described with reference to the drawings in which:

FIG. 1A shows a schematic representation of a proposed double ring electrode structure and measurement system that allows individual monomer detection through resonant tunneling during electrode voltage scan.

FIG. 1B shows a cross-sectional view of a double-ring electrode structure and measurement system.

FIG. 2A shows a schematic representation of a second embodiment of the present invention.

FIG. 2B shows a cross sectional view of the second embodiment of the present invention.

FIG. 3A shows a third embodiment of the present invention.

FIG. 3B shows a cross section of the third embodiment of the present invention.

FIG. 4 shows a fourth embodiment of the present invention.

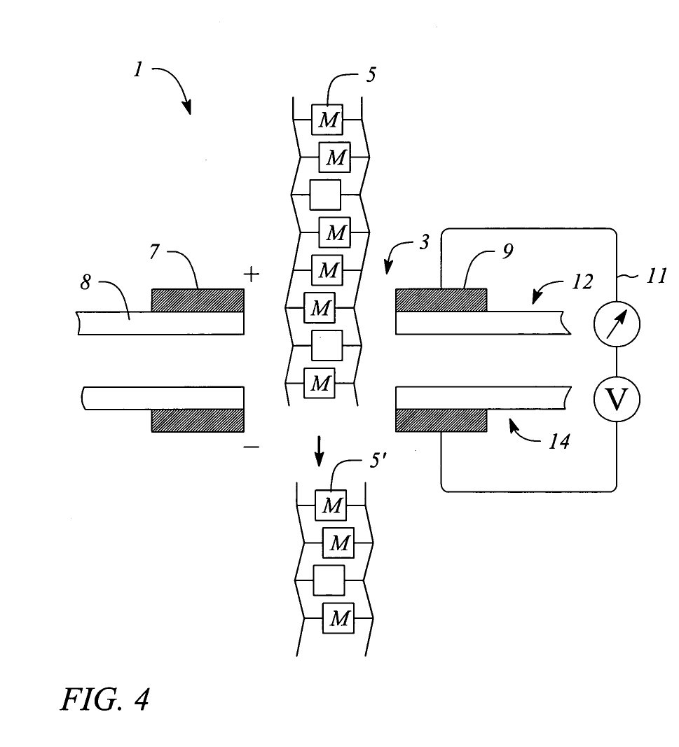

FIG. 5 shows a flow chart of the steps of the method of the present invention.

### DETAILED DESCRIPTION OF THE INVENTION

Before describing the present invention in detail, it is to be understood that this invention is not limited to specific compositions, process steps, or equipment, as such may vary. It is also to be understood that the terminology used herein is for the purpose of describing particular embodiments only, and is not intended to be limiting.

It must be noted that, as used in this specification and the appended claims, the singular forms “a”, “an” and “the” include plural referents unless the context clearly dictates otherwise. Thus, for example, reference to “a probe” includes more than one probe, reference to “a target” includes a plurality of targets and the like.

In describing and claiming the present invention, the following terminology will be used in accordance with the definitions set out below.

A “biopolymer” is a polymer of one or more types of repeating units. Biopolymers are found in biological systems and particularly include peptides and polynucleotides, as well as such compounds composed of or containing amino acid or nucleotide analogs or non-nucleotide groups. This includes polynucleotides in which the conventional backbone has been replaced with a non-naturally occurring or synthetic backbone, and nucleic acids in which one or more of the conventional bases have been replaced with a synthetic base capable of participating in Watson-Crick type hydrogen bonding interactions. Polynucleotides include single or multiple stranded configurations, where one or more of the strands may or may not be completely aligned with another. While biopolymers of the present invention will typically be double-stranded, this is not essential. Specifically, a “biopolymer” includes DNA (including cDNA), RNA and polynucleotides, regardless of the source.

A “nucleotide” refers to a sub-unit of a nucleic acid and has a phosphate group, a 5-carbon sugar and a nitrogen containing base, as well as analogs of such sub-units.

An “oligonucleotide” generally refers to a nucleotide multimer of about 10 to 100 nucleotides in length, while a “polynucleotide” includes a nucleotide multimer having any number of nucleotides.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid references a liquid containing either a biomonomer or biopolymer, respectively (typically in solution).

The term “doping” shall refer to the process of adding a metal or other conductive molecule or material to a complex, nucleic acid, polymer or biopolymer. The term includes adding the metal to any part or component of the molecules or complexes. The metal or conductive molecule or material need not be added or intercalated between the molecules themselves, but may contact one or more of the molecules in some manner.

The term “initial complex” shall refer to a complex that contains at least one metal, and a least one biopolymer. The complex may or may not be directly attached to a surface or substrate.

The term “voltage source” shall refer to any machine, device, or apparatus for adding a potential to the initial complex. The term is intended to be broad based and include any and all circuitry whether chemical, electrical or mechanical that will provide a potential to the system and final complex. Other means and methods well known in the art are intended to be included in the definition.

The term “aptamer” shall refer to DNA or RNA molecules that have been artificially evolved and selected to bind other molecules, viruses, etc. They have many potential uses in medicine and technology.

The term “derivatives” shall refer to any molecule that can be produced directly from the molecule of interest using synthetic organic chemistry. Derivatives are synthesized molecules that have the original structure modified in some way through the addition or deletion of functional or non-functional groups.

It is to be understood that while the invention has been described in conjunction with the specific embodiments thereof, the foregoing description as well as the example that follows are intended to illustrate and not limit the scope of the invention. Other aspects, advantages and modifications within the scope of the invention will be apparent to those skilled in the art to which the invention pertains.

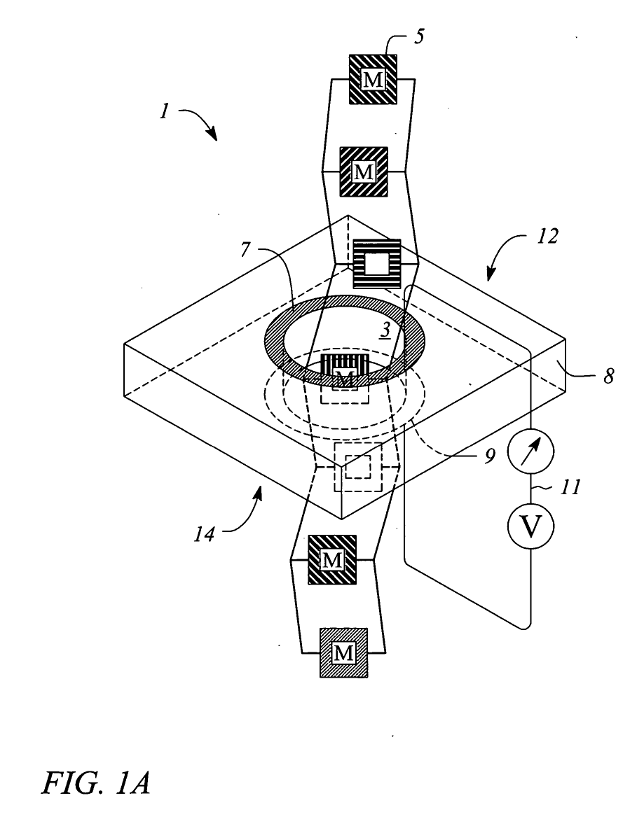

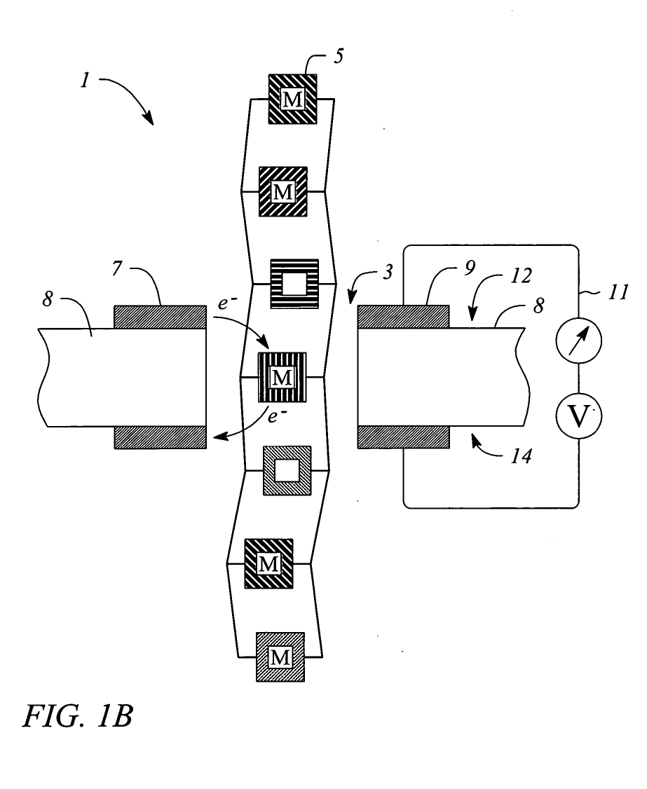

Referring now to FIGS. 1-3, the present invention provides a biopolymer identification apparatus 1 that is capable of identifying or sequencing a biopolymer 5. The biopolymer identification apparatus 1 comprises a first electrode 7, a second electrode 9 and a voltage source 11. Either or both of the electrodes may be ring shaped. The first electrode 7 and the second electrode 9 are electrically connected to the voltage source 11. The second electrode 9 is adjacent to the first electrode 7 and spaced from the first electrode 7. A nanopore 3 may pass through the first electrode 7 and the second electrode 9. However, this is not a requirement of the invention. In the case that the optional substrate 8 is employed, the nanopore 3 may also pass through the substrate 8. Nanopore 3 is designed for receiving a biopolymer 5. When the optional substrate 8 is employed, the first electrode 7 and the second electrode 9 may be deposited on the substrate, or may comprise a portion of the substrate 8. In this embodiment of the invention, the nanopore 3 also passes through the optional substrate 8. Other embodiments of the invention may also be possible where the first electrode 7 and the second electrode 9 are positioned in the same plane (as opposed to one electrode being above or below the other) with or without the optional substrate 8. The use of multiple electrodes and/or substrates are also within the scope of the invention.

The biopolymer 5 may comprise a variety of shapes, sizes and materials. The shape or size of the molecule is not important to the invention, but it must be capable of translocation through the nanpore 3. For instance, both single stranded and double stranded RNA and DNA may be used as a biopolymer 5. In addition, the biopolymer 5 may contain functional groups that are charged. Furthermore, metals or materials may be added, doped or intercalated within the biopolymer 5 to provide a net dipole, a charge or allow for conductivity through the biopolymer.

The first electrode 7 may comprise a variety of electrically conductive materials. Such materials include electrically conductive metals and alloys of tin, copper, zinc, iron, magnesium, cobalt, nickel, and vanadium. Other materials well known in the art that provide for electrical conduction may also be employed. When the first electrode 7 is deposited on or comprises a portion of the solid substrate 8, it may be positioned in any location relative to the second electrode 9. It must be positioned in such a manner that a potential or voltage can be established between the first electrode 7 and the second electrode 9. In addition, the biopolymer 5 must be positioned sufficiently close so that a portion of it may be identified or sequenced. In other words, the first electrode 7, the second electrode 9, and the nanopore 3 must be spaced and positioned in such a way that the biopolymer 5 may be identified or sequenced. This should not be interpreted to mean that the embodiment shown in FIG. 1 in any way will limit spatial orientation and positioning of each of the components of the invention. The first electrode 7 may be designed in a variety of shapes and sizes. Other electrode shapes well known in the art may be employed. In addition, parts or curved parts or rings or other shaped electrodes may be used with the present invention. The electrodes may also be designed in broken format or spaced from each other. However, the design must be capable of establishing a potential or voltage across the first electrode 7, the biopolymer 5 positioned in the nanopore 3, to the second electrode 9.

The second electrode 9 may comprise the same or similar materials as described above for the first electrode 7. As discussed above, its shape, size and positioning may be altered relative to the first electrode 7 and the nanopore 3.

The optional substrate 8 may comprise a variety of materials known in the art for designing substrates or nanopores. The substrate 8 may or may not be a solid material. For instance, the substrate 8 may comprise a mesh, wire, or other material that a nanopore may be constructed. Such materials may comprise silicon, silica, solid-state material such as Si₃N₄, carbon based materials, plastics, metals, or other materials known in the art for etching or fabricating semiconductor or electrically conducting materials. The solid substrate 8 may comprise various shapes and sizes. However, it must be large enough and of sufficient width to be capable of forming a nanopore 3 through it.

The nanopore 3 may be positioned anywhere on/through the optional substrate 8. As described above, the nanopore 3 may also be established by the spacing between the first electrode 7 and the second electrode 9 (in a planar or non-planar arrangement). When the substrate 8 is employed, it should be positioned adjacent to the first electrode 7 and the second electrode 9. The nanopore may range in size from 1 nm to as large as 300 nm. In most cases, effective nanopores for identifying and sequencing biopolymers would be in the range of from 2-20 nm. These size nanopores are just large enough to allow for translocation of a biopolymer 5. The nanopore 3 may be established or designed using any methods well known in the art. For instance, the nanopore 3, may be sculpted in the substrate 8, using argon ion beam sputtering, etching, photolithography, or other methods and techniques well known in the art.

The voltage source 11 may be positioned anywhere relative to the substrate 8, the nanopore 3, the first electrode 7 and the second electrode 9. The voltage source 11 should be capable of ramping to establish a voltage gradient between the first electrode 7 and the second electrode 9. A variety of voltage sources 11 may be employed with the present invention. A number of voltage sources are known in the art. The voltage source 11 has the ability to ramp to establish a voltage gradient between the first electrode 7 and the second electrode 9. This is an important aspect of the present invention and for this reason is discussed in more detail below.

An optional means for signal detection may be employed to detect the signal produced from the biopolymer and voltage source 11. This means for signal detection may be any structure, component or apparatus that is well known in the art and that may be electrically connected to one or more components of the present invention.

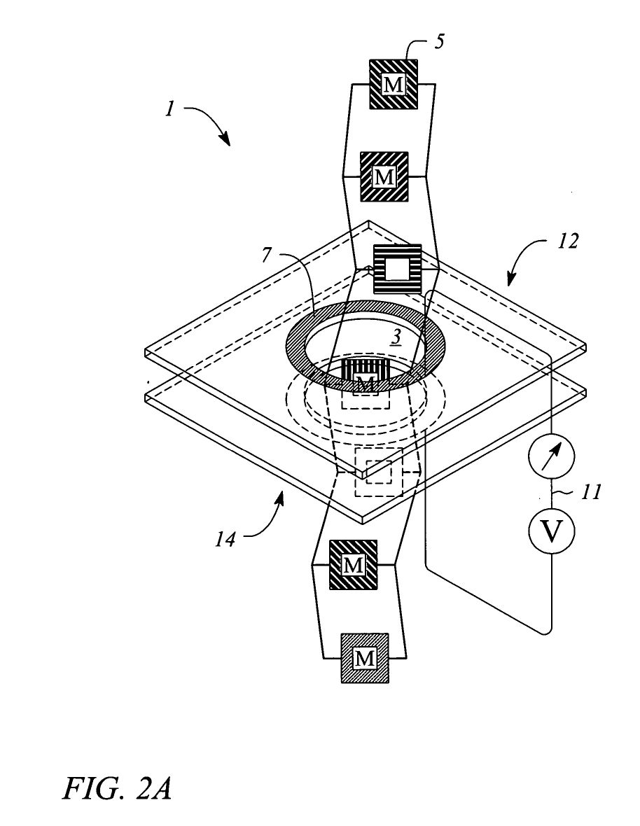

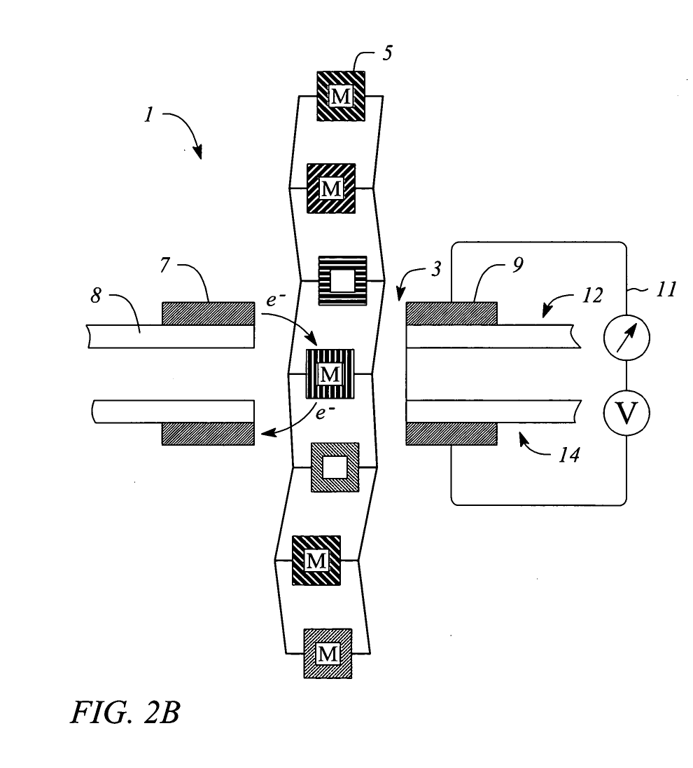

Referring now to FIGS. 2A and 2B, a second embodiment of the invention, a series of separate substrates may be employed. For instance, a first substrate 16 and a second substrate 18 may be employed in place of the single substrate 8. In this embodiment of the invention, the first electrode 7 comprises first substrate 16 or a portion of this substrate. The electrode may be embedded, attached, layered, deposited, etched on the substrate or it may comprise all or a portion of the second substrate 18. The first substrate 16 is positioned adjacent to the second substrate 18. The figure shows the first substrate 16 positioned spatially above the second substrate 18. The first electrode may comprise a first nanopore 3 while the second electrode 9 may comprise a second nanopore 3′. The first nanopore 3 of the first electrode 7 and the second nanopore 3′ of the second electrode 9 may have center points that are coaxially aligned to form a single contiguous pore that the biopolymer 5 may translocate through. It is within the scope of the invention that the nanopore 3 and the nanopore 3′ center points may be offset or spaced at relative angles and distances from each other (Tim, please check the numbering in this paragraph. 16 and 18 should be 12, 14. But you used 14 in other Figure. It is up to you if you prefer to change the number from graph.).

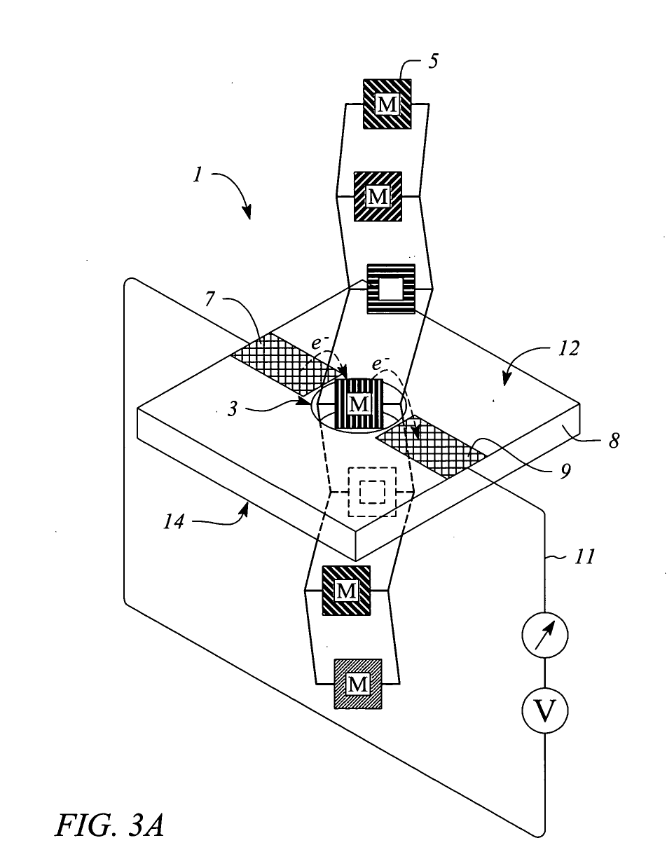

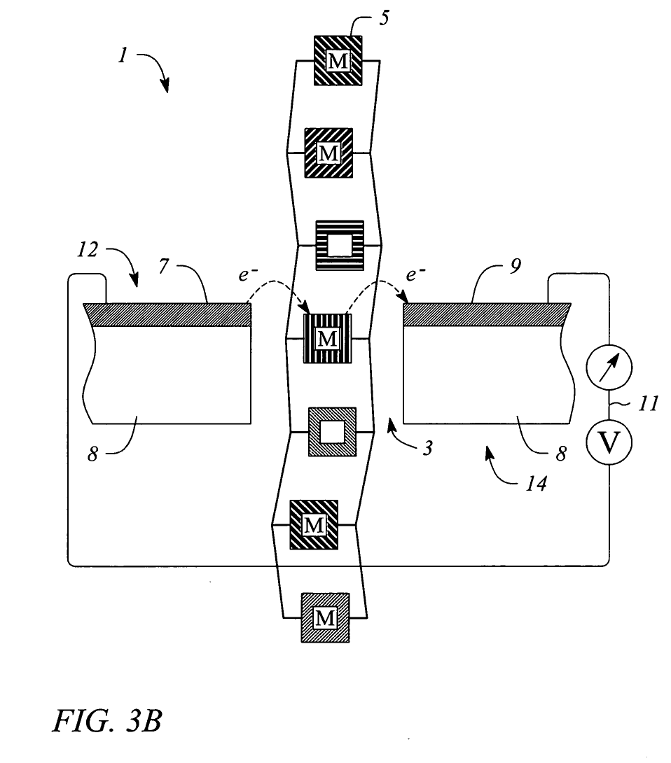

Referring now to FIGS. 3A and 3B, a third embodiment of the present invention is provided. In this embodiment, the first electrode 7 and the second electrode 9 are spaced in the same plane. One or more optional substrates or electrodes may be employed. When the optional substrate 8 is not employed, the first electrode 7 and the second electrode 9 may be positioned adjacent to define the nanopore 3. Although the figures show a pair of electrodes, the invention should not be interpreted to be limited to only this configuration. Various electrodes of varying shapes and sizes may be employed. Furthermore, it is anticipated that the invention comprises a number of similar or different electrodes capable of tunneling in a variety of directions and space (i.e. one, two, and three dimensional space).

An important component of the invention is the voltage source 11. As described above, the voltage source 11 may be ramped. The purpose of the ramping and how it is accomplished is described in detail in application Ser. No. 10/352, 675, filed Jan. 27, 2003, entitled “Apparatus and Method for Biopolymer Identification During Translocation through a Nanopore”, which is herein incorporated by reference in its entirety.

Having discussed the apparatus of the present invention, a description of the method of the present invention is now in order. The method of the present invention comprises first adding a metal to a biopolymer 5 to form an initial complex. The biopolymer is then moved through a nanopore in a substrate and a voltage is ramped across the nanopore to determine the presence of the biopolymer. In certain instances, the ramped voltage may be used for determining the sequence of the biopolymer. In other embodiments, the doping of the biopolymer indicates the presence or absence of base pairing in the biopolymer. In the event that there is no base pair match, an open space is indicated. The metal only incorporates when there is a base pair match. When a base pair match is present (or when there is complete annealing) the metal dopes or intercalates into the biopolymer and makes it conductive. This then allows for electrical conductivity or tunneling between electrodes. For instance, if the nanopore is 10 nm in diameter the electons cannot tunnel between electrodes. However, if the annealed duplex is traversing through the nanopore a 2 nm gap may hypothetically exist from electrode to biopolymer on both sides (the biopolymer hypothetically takes up 6 nm). This configuration would then allow for electron movement from electrode to biopolymer to electrode.

In certain embodiments of the invention the biopolymer may comprise an oligonucleotide. In this embodiment a first oligonucleotide is hybridized to a second oligonucleotide to form an initial complex. Next, a metal is added to the initial complex to form a final complex. A ramped voltage is then applied to the final complex to produce a detectable signal.

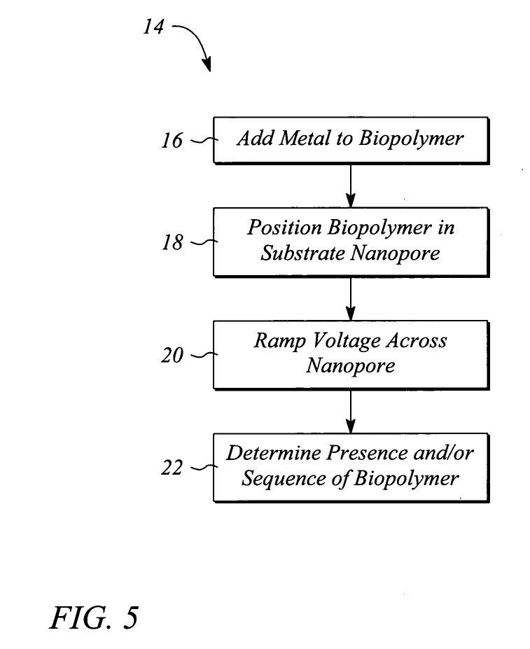

FIG. 5 shows a flow chart of the steps provided by the method of the present invention. Referring to FIG. 5, the steps of the method of the present invention are generally indicated by reference numeral 14. The first step of the method as shown in FIG. 5 comprises adding the metal to the biopolymer (step shown as reference numeral 16). The metal may be added to the biopolymer by solution or any other methods that allow the metal to bind or interact with the biopolymer. Generally, this type of addition is accomplished through non-specific binding. The metals bind the biopolymers based on mass to charge ratio. The biopolymers in most instances will be hybridized together. For instance, the biopolymer may be one or more oligonucleotides that bind together through hybridization. After the metal has been inserted into the double helical structure of biopolymer, the biopolymer is positioned in the nanopore in the substrate (step shown as reference numeral 18). This positioning may be static or dynamic. In other words, the biopolymer may be positioned and then the sequence read or the biopolymer may be moved or threaded through the nanopore of the substrate. Concomittantly, the biopolymer is then translocated across the nanopore and the voltage is ramped across the nanopore (step shown as reference numeral 20). The presence, quantity and/or absence of metal can be determined. This results in a signal indicative of the base pairs and metals positioned in the center portion of the nanopore between the first electrode and the second electrode. The presence and/or sequence of the biopolymer can then be determined (step shown as reference numeral 22) based on the detection of metal in the structure of biopolymers. M-DNA cannot be formed between unmatched base pair (Jeremy Lee et al. WO 99/31115). The known probe polynucleotide sequence is thus indicative to the target sequence and the position for the occurrence of mismatched pair.

1. A method for detecting the presence of a biopolymer, comprising:
(a) adding a metal to a biopolymer;
(b) positioning the biopolymer in a nanopore in a substrate; and
(c) ramping a voltage source across the nanopore in the substrate to produce a detectable signal.

2. A method as recited in claim 1, wherein said metal for doping said biopolymer is selected from the group consisting of zinc, nickel and cobalt.

3. An apparatus as recited in claim 1, wherein said biopolymer is conductive.

4. An apparatus as recited in claim 1, wherein biopolymer is a double stranded oligonucleotide.

5. A method for detecting the presence of an oligonucleotide, comprising:
(a) hybridizing a first oligonucleotide to a second oligonucleotide;
(b) adding a metal to the hybridized oligonucleotides to form an initial complex; and
(c) applying a ramped voltage to the initial complex to produce a detectable signal.

6. A method as recited in claim 5, wherein the metal added in step (b) is selected from the group consisting of zinc, cobalt and nickel.

7. A method as recited in claim 5, wherein said biopolymer is a nucleic acid.

8. A method as recited in claim 7, wherein said nucleic acid is selected from the group consisting of RNA, DNA, aptamers and their derivatives.

9. A method as recited in claim 5, wherein a plurality of metal is added to said initial complex.

10. A method as recited in claim 5, wherein a plurality of different metals are added to said initial complex.

11. A method as recited in claim 5, wherein said initial complex is conductive.
